(19) United States
(12) Patent Application Publication
(10) Pub. No.: US 2010/0190214 A1
(43) Pub. Date: Jul. 29, 2010

(54) BIOCHEMICAL TREATMENT APPARATUS AND METHOD COMPRISING LIQUID HANDLING MECHANISM

(75) Inventors: Ikumasa Ikeda, Miura-gun (JP); Hideaki Okamoto, Yokohama-shi (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/752,012

(22) Filed: Mar. 31, 2010

Related U.S. Application Data
(62) Division of application No. 11/519,964, filed on Sep. 13, 2006.

(30) Foreign Application Priority Data
Oct. 4, 2005 (JP) 2005-291181

(51) Int. Cl. C12P 19/34

(52) U.S. Cl. 435/91.2

(57) ABSTRACT

Nucleic acid solution, magnetic particles solution and primer or eluate are moved between a lot of wells provided in reaction/storage vessel 3 by pipette device 40 to perform the first PCR, purification and the second PCR. In the time point where the first PCR and purification end, pipette tip 42 of pipette device 40 is exchanged and the second PCR is performed using new pipette tip 42.

### BACKGROUND OF THE INVENTION

1. Field of the Invention

The present invention relates to a biochemical treatment apparatus and method for performing amplification of nucleic acid and the like, particularly a biochemical treatment apparatus and method comprising a liquid handling mechanism.

2. Description of the Related Art

As for processes to quickly and precisely perform analysis of base sequence in a nucleic acid and detection of target nucleic acid in a nucleic acid sample, there have been proposed a number of processes which utilize a hybridization reaction using a probe carrier represented by DNA microarray. A DNA microarray is a device in which probes having base sequences complementary to target nucleic acid are immobilized on a solid phase such as a bead or a glass plate at high density. A detection method of the target nucleic acid which uses this DNA microarray generally has the following steps.

First, the target nucleic acid is amplified by an amplification process represented by the PCR (polymerase chain reaction) process. Specifically, first and second primers are added to the nucleic acid sample first and subjected to a predetermined temperature cycle. This allows the first primer to be specifically bounded to a part of the target nucleic acid and the second primer to be specifically bounded to a part of a nucleic acid which is complementary to the target nucleic acid. When a double strand nucleic acid containing the target nucleic acid and the first and second primers are bound, the double strand nucleic acid containing the target nucleic acid is amplified by elongation reaction. This treatment is the first amplification step, and it is called the first PCR.

Next, after the double strand nucleic acid containing the target nucleic acid is amplified enough, substances other than the amplified double strand nucleic acid, such as unreacted primers and fragment of nucleic acids, are removed by purification. For the purification method, a method of adsorbing the double strand nucleic acid on magnetic particles or a method of adsorbing the double strand nucleic acid to a column filter is used.

Then, a third primer is added to the nucleic acid sample purification of which is completed and a temperature cycle is conducted. The third primer is labeled with an enzyme, a fluorescent substance or a luminescent substance, etc. and specifically bound to a part of a nucleic acid complementary to the target nucleic acid. When the third primer binds with a nucleic acid complementary to the target nucleic acid, the target nucleic acid labeled with an enzyme, a fluorescent substance or a luminescent substance, etc. is amplified by elongation reaction. This treatment is the second amplification step, and it is called the second PCR. It is also known as another labeling method in the second PCR to label a target nucleic acid using a fluorescence-labeled nucleotide as substrate.

When the above-mentioned first PCR, purification and the second PCR are performed as the first step, and, as a result, the target nucleic acid is contained in the nucleic acid sample, a labeled target nucleic acid is generated. However, when the target nucleic acid is not contained in the nucleic acid sample, a labeled target nucleic acid is not generated.

Next, this nucleic acid sample is exposed to a DNA microarray as the second step and subjected to hybridization with probes on the DNA microarray. When the nucleic acid sample contains a target nucleic acid complementary to a probe, the probe and the target nucleic acid form a hybrid body.

Then, as the third step, detection of the target nucleic acid is performed. Whether the probe and the target nucleic acid form a hybrid body or not can be detected by the labeling substance of target nucleic acid as above and this enables to confirm the presence of a specific base sequence.

Japanese Patent Application Laid-Open No. H07-107999 discloses a device which can process the first to third steps continually in one device. This device has a movable dispenser, and has a construction to transport necessary liquids to each container and react them.

In addition, some reagents used in a biochemical reaction apparatus should be prevented from deterioration. Inter alia, since enzymes important to perform a biochemical reaction may be deteriorated at normal temperature and cannot be used for biochemical reaction, such a device needs a refrigerating section which keeps enzymes at a low temperature.

In the above-mentioned first step, the target nucleic acid is exponentially amplified in the first PCR, and only the labeled target nucleic acid is amplified in the second PCR. In this case when the second PCR is laced with the first or second primer used for the first PCR, the exponential amplification similar to the first PCR is resulted again in the second PCR. As a result, the following problems occur.

As the first problem, the target nucleic acid which is not labeled is increased by the first primer which is not labeled in the second PCR. Since this target nucleic acid which is not labeled binds with the probe array, binding with the probe array of the labeled target nucleic acid which is originally aimed is obstructed.

As the second problem, nucleic acids complementary to the target nucleic acid are amplified in the second PCR in the same way as in the first PCR, and the nucleic acids complementary to the target nucleic acid increase. When nucleic acids complementary to the target nucleic acid increase in the nucleic acid sample, it becomes hybridization condition in the liquid with high hybridization efficacy, and the increased complementary nucleic acids is bound to the labeled target nucleic acid. As a result, the labeled target nucleic acid in the form of a single strand in the nucleic acid sample decreases, and therefore the binding of the labeled target nucleic acid to the probe array is decreased.

### SUMMARY OF THE INVENTION

Accordingly, an object of the present invention is to provide a biochemical treatment apparatus with little contamination.

The biochemical treatment process of the present invention comprises the following steps: a first amplification step in which a target nucleic acid and a nucleic acid complementary to the target nucleic acid are amplified; a purification step in which substances other than the desired nucleic acid amplified in the above first amplification step are removed; a second amplification step in which a labeled target nucleic acid is amplified using the above purified nucleic acids; and a step of exchanging or cleaning at least a part of a liquid handling mechanism for transporting a liquid containing the above nucleic acids which part is in contact with the above liquid between the above first amplification step and the second amplification step.

In addition, biochemical treatment apparatus of the present invention comprises the following: a first amplification treatment section in which a target nucleic acid and a nucleic acid complementary to the target nucleic acid are amplified; a purification treatment section in which substances other than the desired nucleic acids amplified in the above first amplification step are removed; a second amplification treatment section in which a labeled target nucleic acid is amplified using the above purified nucleic acids; a liquid handling mechanism for transporting a liquid containing the above nucleic acids between the above first amplification treatment section and the second amplification treatment section; and a section of exchanging or cleaning at least a part of the above liquid handling mechanism which part is in contact with the above liquid.

Further features of the present invention will become apparent from the following description of exemplary embodiments with reference to the attached drawings.

### BRIEF DESCRIPTION OF THE DRAWINGS

FIGS. 1A, 1B and 1C are outlined plan views of a biochemical treatment apparatus of the present invention.

FIG. 2 is a perspective view showing a reaction/storage vessel of the apparatus shown in FIGS. 1A, 1B and 1C.

FIG. 3 is a front elevation view of an amplifying section of the apparatus shown in FIGS. 1A, 1B and 1C.

FIG. 4 is a plan view of an amplifying section shown in FIG. 3.

FIG. 5 is a front elevation view showing the state of the amplifying section shown in FIG. 3 before starting amplification.

FIG. 6 is a front elevation view showing the state in which a nucleic acid solution is discharged in the amplifying section shown in FIG. 3.

FIG. 7 is an enlarged front elevation view showing a purification step in the amplifying section shown in FIG. 3.

### DESCRIPTION OF THE EMBODIMENTS

Embodiments of the present invention are described below.

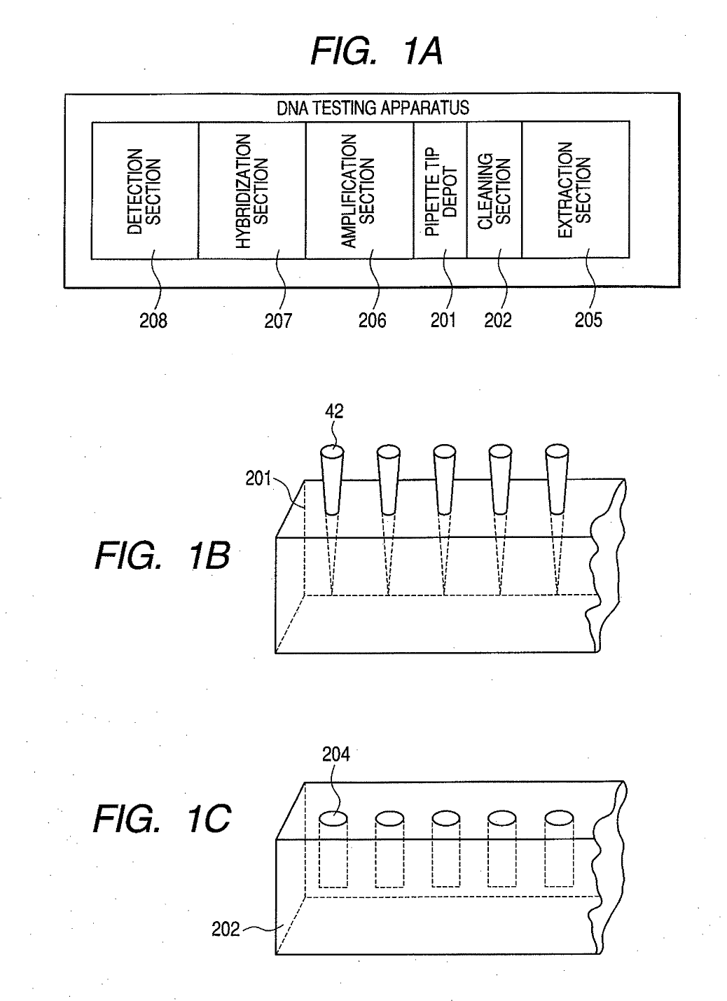

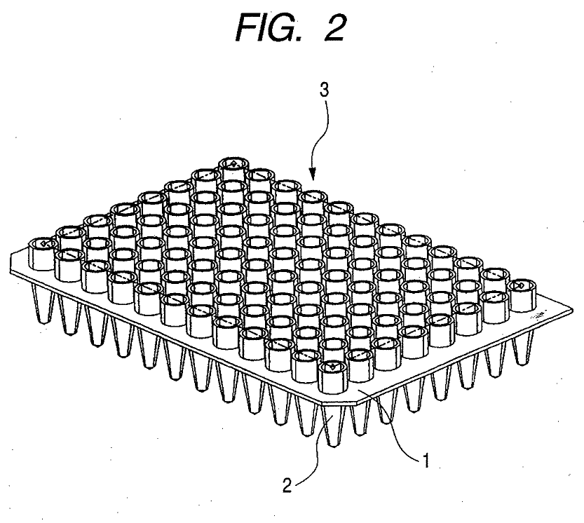

First, the biochemical treatment apparatus of the present invention is described by way of a DNA testing apparatus. FIGS. 1A, 1B and 1C show outlines of the basic constitution thereof. The DNA testing apparatus of this embodiment can carry out an amplification step, a hybridization step and a detection step for nucleic acids in one apparatus sequentially. This DNA testing apparatus comprises a reaction/storage vessel 3 (cf. FIG. 2) and a main body. An example of amplifying section 206 (cf. FIG. 1A) of the main body of the apparatus generally consists of a thermal cycle section, a purification section and a refrigerating section. The thermal cycle section, purification section and refrigerating section are in the vicinity of a reaction/storage vessel 3.

Firstly describing the reaction/storage vessel 3, this is composed of a commercial PCR microplate 1 having 96 wells or an article in which wells are formed in a similar configuration. This reaction/storage vessel 3 is filled with reagents used for PCR beforehand. One side of reaction/storage vessel 3 comprises wells for the first PCR and the second PCR, the central part of the vessel comprises wells for the purification step, and the other side of the vessel comprises wells filled with reagents used for the purification step, the first PCR and the second PCR.

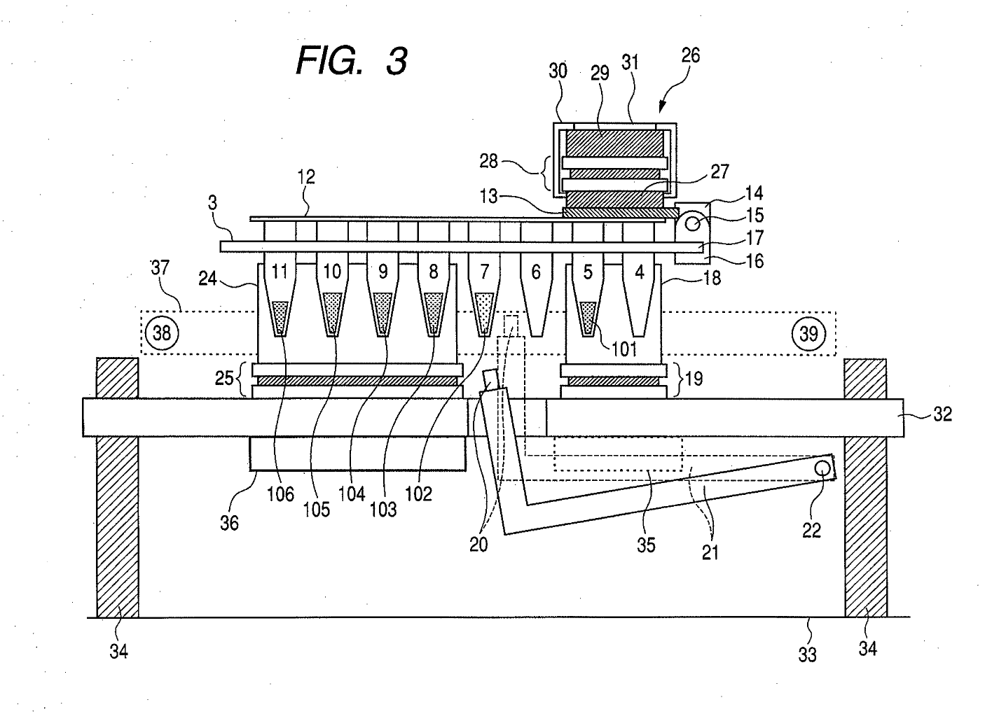

The thermal cycle section of amplifying section 206 consists of a thermal cycle block 18 (cf. FIGS. 3, 4, 5 and 6) comprising a plurality of concaves, formed of a metal with good thermal conductivity such as aluminum or a copper alloy and capable of closely fitting with the reaction/storage vessel 3, Peltier elements, a heater, etc. The concaves are formed in the same number as that of the wells (wells 4 and 5 in the example shown in FIGS. 3, 4, 5 and 6) in which the first PCR and the second PCR are performed. The first PCR and the second PCR are performed in the condition where this thermal cycle block 18 is fit with reaction/storage vessel 3. Generally, temperature cycles of keeping temperatures of about 92° C.-55° C.-72° C. for predetermined period of time respectively are repeated by around 40 times in the first PCR, and the temperature cycles are repeated by around 25 times in the second PCR treatment.

The thermal cycle section has a heating unit 26 (cf. FIG. 3) above the reaction/storage vessel 3. This heating unit 26 comprises a heating block 27 which consists of a metal with good thermal conductivity such as aluminum or a copper alloy, Peltier element 28, a heater, etc. Heat is given to the heating block 27 and heats the reaction/storage vessel 3 from the upper part. The heating block 27 is configured in a size covering only the upper part of the wells in which PCR is performed during the first PCR and the second PCR and not heating the refrigerating section. Since the temperature of the inner wall surface of the wells in which PCR is performed is elevated by this heating block 27, dew condensation caused by vapor of the solution which has been evaporated and comes up and adhere onto the inner wall surface can be prevented.

The purification section of amplifying section 206 is provided at the position facing the central part of reaction/storage vessel 3. Purification is generally performed by using magnetic particles filled in the wells beforehand and magnet 20 arranged around the wells. After nucleic acid solution, cleaning fluid and ethanol are supplied separately into the wells (wells 6 and 7 in the example shown in FIGS. 3, 4, 5 and 6) of purification section, they are stirred to allow the nucleic acid to be adsorbed on the magnetic particles. Then the magnetic particles with nucleic acid adsorbed thereon are held at one position by bringing magnet 20, which is usually held at a position remote from the wells, close to the wells in the purification section, and the solution in the wells is aspirated while leaving the magnetic particles with nucleic acid adsorbed thereon in the wells. Then, eluant is supplied in these wells to separate the nucleic acid from the magnetic particles and while the magnetic particles are held at one position by bringing magnet 20 close to the wells in the purification section, the solution other than the magnetic particles in the wells is aspirated. Purified nucleic acid solution is obtained by such a step.

The refrigerating section is provided at a position facing a side of the reaction/storing vessel opposite to the side facing the thermal cycle section. The refrigerating section comprises a cooling block 24 comprising a plurality of concaves, formed of a metal with good thermal conductivity such as aluminum or a copper alloy and capable of closely fitting with the reaction/storage vessel 3 and Peltier element 25, etc. (cf. FIGS. 3, 4, 5 and 6). The concaves are formed at least in the same number as that of the wells (wells 8 to 11 in the example shown in FIGS. 3, 4, 5 and 6) filled with reagent. Cooling block 24 is covered with an insulative material from around.

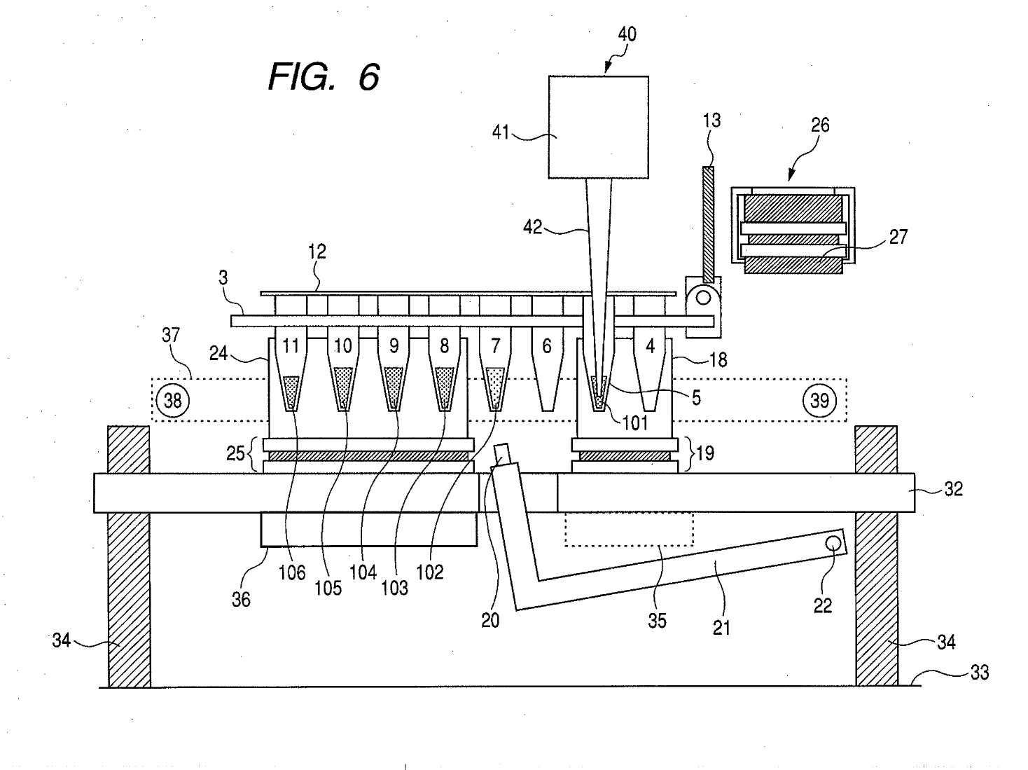

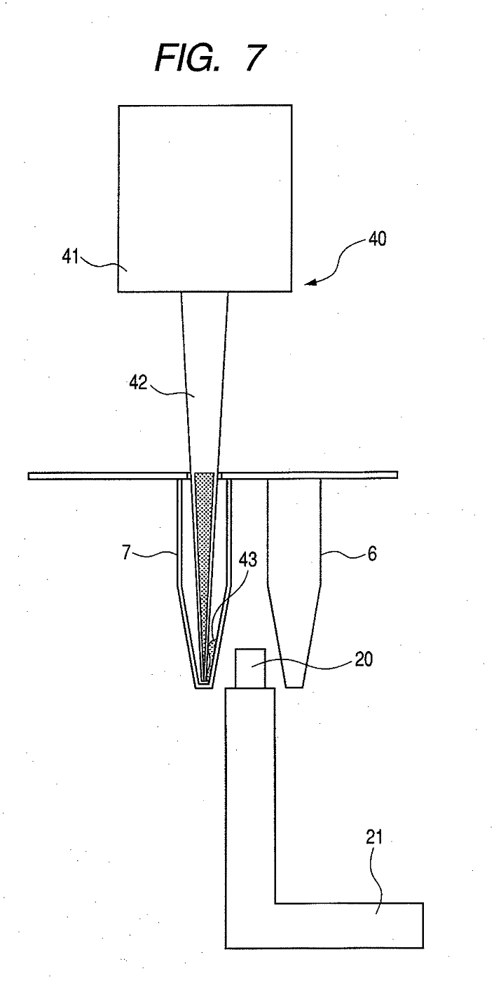

In this embodiment, pipette device 40 (cf. FIGS. 6 and 7) moving a solution among the wells 4-11 which face the above-mentioned thermal cycle section, refrigerating section and purification section respectively is provided. The pipette device 40 has a pipette part 41 and a removable pipette tip 42 at its end, and the pipette tip 42 is exchanged as required.

Refrigerating section keeps a reagent at a temperature which does not deteriorates the reagent after reaction/storage vessel 3 is set at the main body of the apparatus until it is actually used. The storage temperature of a reagent is in the range of around 4° C. to 20° C. and this temperature should be maintained without being affected by the temperature (55° C. to 92° C.) of the thermal cycle section. Therefore, it is configured with the distance between the refrigerating section and the thermal cycle section lengthened by providing the purification section between the refrigerating section and the thermal cycle section so that the temperature of each section does not influence mutually. Since the limited size of PCR microplate 1 is used effectively by arranging the thermal cycle section, purification section, refrigerating section in this order, the DNA testing apparatus can be made compact. Furthermore, the thermal cycle section, purification section and refrigerating section are located very closely so that even one reaction/storage vessel 3 can entirely cover them all without moving the reaction/storage vessel 3. Accordingly, transfer distance of pipette device 40 which supplies and aspirates solutions can be shortened.

Next, specific examples of the present invention are described in detail with reference to the drawings.

FIG. 1A is an outline of a DNA testing apparatus of this example. The DNA testing apparatus has extraction section 205 to extract DNA from a biological sample, amplifying section 206 to amplify the DNA, hybridization section 207 to hybridize the amplified DNA with probe DNA and detection section 208 to detect the presence of hybridization bond. Analyzing process proceeds in the order of extraction section 205, amplifying section 206, hybridization section 207, detection section 208 in this DNA testing apparatus. Furthermore, pipette tip depot 201 for pipette tip 42 of pipette device 40 which handles liquid between each of the sections 205 to 208 and in each of the sections 205 to 208 in each step is provided as shown in FIG. 1B. A plurality of pipette tip 42 are ready for use in pipette depot 201 as shown in FIG. 1B, and they are picked out with pipette part 41 and used as required, and after used, they are returned to pipette depot 201 or disposed into an area for disposal (not illustrated). In this example, pipette tip 42 is an exchangeable-type tip as described later. However, a permanent-type liquid dispensing unit can be also used while cleaning is performed. In that case, cleaning section 202 to wash pipette tip 42 is provided as shown in FIG. 1C. This cleaning section 202 is illustrated in FIG. 1A, but as described later, it is not necessary when pipette tip 42 is exchanged before the second PCR. It is provided when pipette tip 42 is used repeatedly by cleaning without changing.

FIG. 2 is a perspective view showing the well site of reaction/storage vessel 3. Although not shown in FIG. 1A, this reaction/storage vessel 3 is set in the main body of the apparatus to constitute the DNA testing apparatus. The well site of reaction/storage vessel 3 is composed of commercially available FCR microplate 1 made of polypropylene or an article in which wells are formed in a similar configuration, and 96 wells are arranged in the shape of 8×12 matrix with a pitch of 9 mm. The point (bottom) 2 of each well is in such a shape as fit with the thermal cycle block and the cooling block mentioned later.

FIG. 3 is a view in which amplifying section 206 of the DNA testing apparatus is viewed from the front side and shows the structure of reaction/storage vessel 3 and the sequence of the wells as well as positional relationship of amplifying section 206 with the thermal cycle section, refrigerating section and purification section. Condition in the first PCR and the second PCR is shown in FIG. 3.

Reaction/storage vessel 3 uses 8×12 wells dividing them by 8 wells into 12 lines and 12 samples can be analyzed at the same time. Well 4 positioned most right in FIG. 3 is a well to perform the second PCR and it is empty at first. Next well 5 is a well to perform the first PCR, and filled with solution 101 of an enzyme reagent, primer or the like to be used in the first PCR beforehand. Wells 6 and 7 are purifying wells, and well 7 is used first, and well 6 is used after that. Well 7 is filled with magnetic particles solution 102 beforehand, and well 6 is empty at first. And well 8 is filled with cleaning fluid 103, well 9 is filled with ethanol 104 and well 10 is filled with eluant 105 respectively. Well 11 positioned most left in the drawing is filled with solution 106 such as reagent or primer to be used in the second PCR beforehand. 12 lines comprising eight such combinations of wells are arranged in the direction perpendicular to the drawing plane.

The openings of the 96 wells are sealed by adhering or welding laminate film 12 which consists of aluminum and the like. Contamination of the well inside by foreign materials can be prevented by this. Before a solution is aspirated from the well or before a solution is injected into the well, laminate film 12 is perforated by a cutter part (not illustrated).

Lid 13 made of silicone rubber and lid holding member 14 are pivotably held on laminate film 12 on wells 4 and 5 by supporting axis 15. Lid 13 is configured in a size to cover only 12 lines of wells 4 and 5 (24 well in total), and this is the same size as that of the heating block mentioned later. A torsion coiled spring (not illustrated) is attached to supporting axis 15 to force lid 13 and lid holding member 14 in the direction to shut wells 4 and 5 tightly. Supporting axis 15 is rotatably held with bearing part 16. When a commercial PCR microplate 1 is used as well site, bearing part 16 is fixed to edge 17 of PCR microplate 1 with an adhesive or a screw. When a commercial PCR microplate 1 is not used, the bearing part and a member corresponding to PCR microplate 1 may be integrated.

Thermal cycle block 18 provided in the thermal cycle section consists of a metal with a good thermal conductivity such as aluminum or a copper alloy and is covered with an insulative material (not illustrated) formed of a resin from around so that heat may not escape to the circumference. 24 wells (holes) are formed in this thermal cycle block 18. Thermal cycle block 18 faces with wells 4 and 5, and the inner wall surface of wells of thermal cycle block 18 has a dimension to closely contact with the outside wail surface of wells 4 and 5. Peltier element 19 to heat thermal cycle block 18 is provided below thermal cycle block 18. Peltier element 19 is arranged in the number so that 12 lines of all the wells on the reaction/storage vessel 3 can be heated uniformly. Grease (not illustrated) is applied on the contacting face of Peltier element 19 and thermal cycle block 18 so that they can completely contact with each other to surely transmit heat. A similar effect can be obtained by using a sheet material with a good thermal conductivity in place of grease.

Magnet 20, magnet fixing material 21 and magnet pivotably supporting point 22 are arranged between wells 6 and 7. There are 12 members respectively for magnet 20 and magnet fixing material 21 in correspondence with each line of the wells, and they are rotatably held on magnet pivotably supporting point 22 respectively. Magnet pivotably supporting point 22 is held by the bearing means (not illustrated) formed in holding plate 32 mentioned later. Magnet fixing material 21 is connected with solenoid (not illustrated) at the end on the side opposite to magnet 20. When magnetic particles are to be collected in purification step, electric current is applied to the solenoid to centralize magnetic particles on the wall surface of the wells, which draws in magnet fixing material 21 and allows to magnet 20 to come up. As a result, magnet 20 and magnet fixing material 21 move from the solid line position to the broken line position in FIG. 3.

Cooling block 24 provided in the refrigerating section consists of a metal with a good thermal conductivity such as aluminum or a copper alloy and is covered with an insulative material (not illustrated) formed of a resin from around so that it is hard to be affected by ambient temperature. 48 wells (holes) are formed in this cooling block 24. Cooling block 24 faces with wells 8 to 11, and the inner wall surface of wells of cooling block 24 has a dimension to closely contact with the outside wall surface of wells 8 to 11. Peltier element 25 to cool cooling block 24 is provided below cooling block 24. Peltier element 25 is arranged in the number so that 12 lines of all the wells on the reaction/storage vessel 3 can be cooled uniformly. Grease (not illustrated) is applied on the contacting face of Peltier element 25 and cooling block 24 so that they can completely contact with each other to surely transmit heat. A similar effect can be obtained by using a sheet material with a good thermal conductivity in place of grease.

Heating unit 26 is provided above reaction/storage vessel 3. Heating block 27 of heating unit 26 consists of aluminum or a copper alloy and is configured in a size covering only the upper part of wells 4 and 5 and not heating wells 6 to 11. Peltier element 28 is provided above heating block 27 and grease (not illustrated) is applied on the contacting face of Peltier element 28 and heating block 27 so that they can completely contact with each other to surely transmit heat. In addition, cooling block 29 formed of a metal material is fixed above Peltier element 28 with grease applied on the contacting face in the same construction as above. Cooling block 29 has a hollow structure having an inlet and an outlet and has a structure to allow cooling water to flow through pipes (not illustrated) which are connected to the inlet and the outlet. When heat is released from the backside of Peltier element 28, this cooling block 29 promotes cooling.

Heating block 27, Peltier element 28 and cooling block 29 are held and thermally insulated by holding member 30 which has opening 31 on the top surface. This heating block 27, Peltier element 28, cooling block 29 and holding member 30 constitute one unit (top unit 26) and can be moved to right and left directions in FIG. 3 by driving means (not illustrated). This top unit 26 is configured in a width so that it can heat 12 lines of all the wells on the reaction/storage vessel 3 uniformly, and located at a position to closely contact with wells 4 and 5 of reaction/storage vessel 3 during amplification so as to heat them from the upper part.

Holding plate 32 which hold the thermal cycle section, refrigerating section and purification section as a whole is provided below Peltier elements 19 and 25. Holding plate 32 can be moved to upper and lower directions in FIG. 3 with the thermal cycle section, refrigerating section and purification section as one body driven by leading screw 34 which is supported through an bearing (not illustrated) by base 33 of the main body of the apparatus, and a motor and a driving force transporting system (not illustrated).

Reaction/storage vessel 3 can be pressed against top unit 26 by fitting reaction/storage vessel 3 with thermal cycle block 18 and cooling block 24 and elevating this holding plate 32. This improves close contact between the circumference of the wells of reaction/storage vessel 3 and thermal cycle block 18 and cooling block 24 and thermal conductivity to the wells.

Water-cooling blocks 35 and 36 formed of a metal material are fixed below the lower surface of holding plate with grease applied on the contacting face. Water-cooling blocks 35 and 36 have a hollow structure having an inlet and an outlet and have a structure to allow cooling water to flow through pipes (not illustrated) which are connected to the inlet and the outlet. Water-cooling blocks 35 and 36 are fixed at positions facing Peltier elements 19 and 25 respectively and promote cooling from the lower surface of Peltier elements 19 and 25. Water-cooling block 35 provided below Peltier element 19 is located in such a positional condition that does not cause interference with magnet fixing material 21 and is shown with a dotted line in FIG. 3.

Carriage 37 in which reaction/storage vessel 3 is set is schematically shown with a dotted line in FIG. 3. That is, a height of carriage 37 at which reaction/storage vessel 3 is set in carriage 37 when carriage 37 is at a front position in the apparatus is shown in this FIG. 3. Carriage 37 is driven by leading screw 38 arranged in the direction perpendicular to the drawing plane and a motor and a driving force transporting system (not illustrated) and can be moved forward and backward along guide shaft 39. When carriage 37 is driven backward in the apparatus and reaction/storage vessel 3 reaches the position facing the thermal cycle section, refrigerating section and purification section, it stops. Holding plate 32 is at a position which is lower than the position shown in FIG. 3 and does not contact with reaction/storage vessel 3. Then, holding plate 32 is elevated to a position shown in FIG. 3 with leading screw 34.

DNA testing apparatus is provided with pipette device 40 shown in FIGS. 6 and 7. Pipette device 40 consists of pipette part 41 to supply, discharge and stir the solution, pipette tip 42 removably attached to the end of pipette part 41, a motor and a leading screw (not illustrated) which is a pipette transporting section. Pipette part 41 is configured so that it can move to upper and lower, forward and backward and right and left by pipette transporting section (not illustrated). Furthermore, a cutter part (not illustrated), pipette depot 201 (cf. FIGS. 1A and 1B) and the pipette disposal section (not illustrated), etc. are provided. This cutter part is located at the right side of pipette tip 42 in FIG. 6, can be transported to an appointed position by pipette transporting section and it perforates laminate film 12 which seals the wells tightly. Pipette depot 201 is located around pipette and maintains unpipette tip after use 42 as described above. The pipette disposal section accommodates waste pipette tip 42.

Next, action of this DNA testing apparatus is described. The action of one line of the wells (for one sample) is described in the following description, but other lines work equally, too.

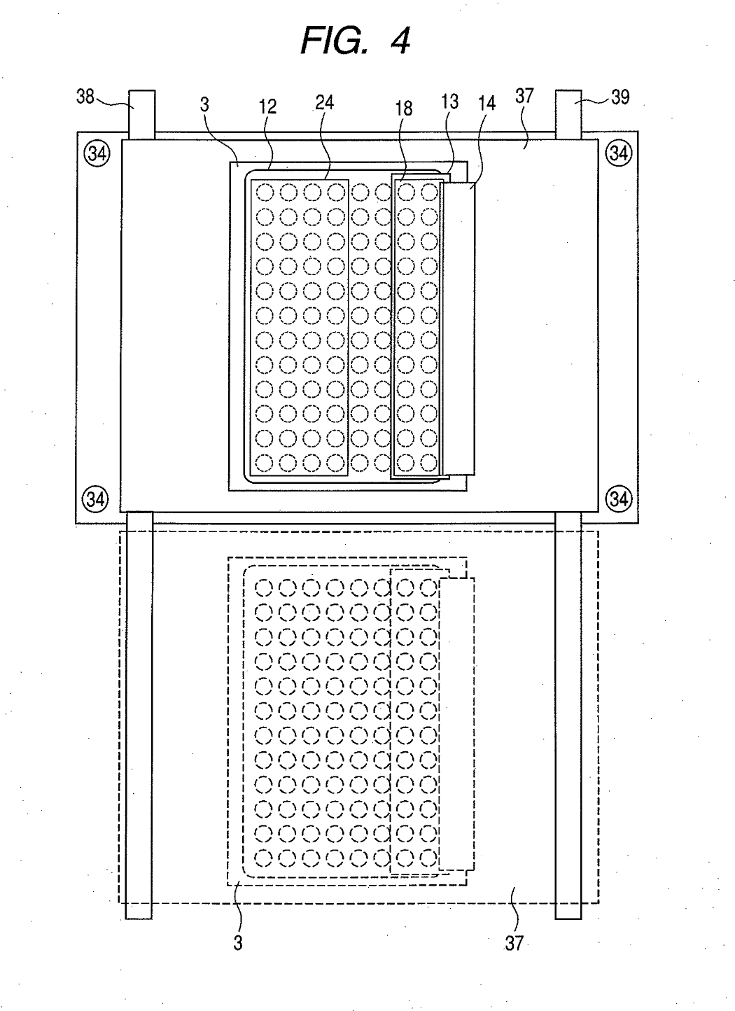

FIG. 4 is a plan view showing an essential part of DNA testing apparatus shown in FIG. 3 except top unit 26 and the like.

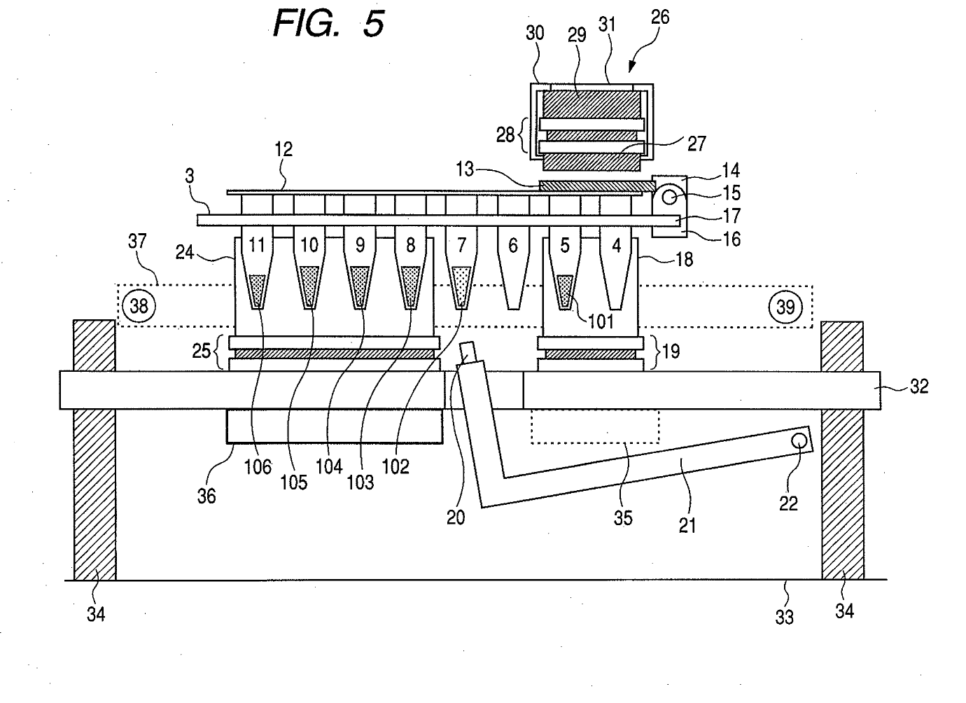

When an operator sets reaction/storage vessel 3 in carriage 37 at a position shown by a broken line of FIG. 4, a driving motor (not illustrated) and leading screw 38 transport reaction/storage vessel 3 backward (upper part of FIG. 4). When carriage 37 reaches the position shown by a solid line in FIG. 4, supporting plate 32 is elevated by leading screw 38 as shown in FIG. 5. The state shown in this FIG. 5 is a state wherein supporting plate 32 is slightly lower than the state shown in FIG. 3, and heating block 27 and lid 13 are not in contact.

A solution containing the nucleic acid which has been already extracted from a biological sample such as blood or urine in extraction section 205 (cf. FIG. 1A) by a well-known method is moved to well 5. Pipette device 40 (cf. FIGS. 6 and 7) is used for transferring the nucleic acid solution. FIG. 6 shows the state in which the nucleic acid solution is discharged into well 5 with pipette device 40 in this way.

First, prior to discharging the nucleic acid solution into well 5, top unit 26 and lid 13 are moved by driving mechanism (not illustrated) to the right side in FIG. 6 respectively and turned about 90° and the upper part of wells 4 and 5 are made open. Then, the part of laminate film 12 facing well 5 is perforated with a cutter part so that pipette tip 42 is able to dip in well 5. Then pipette tip 42 which has aspirated nucleic acid solution from extraction section 205 on the right side in FIG. 6 is moved to above well 5 and then downward into well 5. When pipette tip 42 penetrates to a predetermined depth in well 5, the nucleic acid solution is discharged by pipette part 41. Then, while pipette tip 42 penetrates in well 5, aspiration and discharging are repeated in predetermined time, and the reagent filled in well 5 beforehand and the nucleic acid solution are stirred so as to mix them enough.

After the mixing is finished, pipette part 41 is evacuated from reaction/storing vessel 3 and top unit 26 and lid 13 are made into a state shown in FIG. 5. And holding plate 32 is elevated with leading screw 34 so that it is in a state shown in FIG. 3. The apparatus has a structure in which lid 13 is pressed with a force of 50 to 100 gf per well or more at this time by forcing holding plate 32 toward top unit 26.

The first PCR (the first amplification step) is started in a state shown in FIG. 3. The first PCR amplifies the nucleic acid in a sample tube by repeating around 40 times temperature cycles of maintaining each of the temperatures of about 92° C.-55° C.-72° C. for a predetermined period of time. The refrigerating section maintains reagents at a temperature at which they do not deteriorate, for example, at around 4° C. in the first PCR. Wells 4 and 5 of the thermal cycle section and wells 8 to 11 of the refrigerating section are covered with an insulative material (not illustrated) at the main parts. Furthermore, wells 6 and 7 of the purification section are arranged between the wells 4 and 5 of the thermal cycle section and wells 8 to 11 of the refrigerating section to secure a distance large enough to some extent. The temperature of the thermal cycle section and the refrigerating section do not affect mutually by such a configuration. The temperature of thermal cycle block 18 may be around room temperature when the first PCR is finished.

Purification step is started after the end of the first PCR. FIG. 7 shows a state in which amplification product is adsorbed on the magnetic particles, which are then collected with magnet 20, and pipette tip 42 aspirates substances other than magnetic particles 43 with nucleic acid adhered thereon.

In this purification step, laminate film 12 covering well 7 is perforated by a cutter part (not illustrated) first in the same way as in the first PCR. And pipette part 41 forces pipette tip 42 to penetrate in well 5 and aspirates the solution (sample solution treated in the first PCP) in well 5. Then the pipette moves, and pipette part 41 forces pipette tip 42 to penetrate in well 7 and discharges the solution aspirated from well 5 in well 7. Next, nucleic acid is adsorbed enough on the magnetic particles by stirring with pipette tip 42 magnetic particles solution 102 filled in well 7 beforehand and the solution which moved from well 5. Then, magnet 20 is elevated to the position shown in FIG. 7 to centralize magnetic particles 43 on which nucleic acid adheres in one point of the inner wall of well V. As shown in FIG. 7, substances other than magnetic particles 43 on which nucleic acid adheres are aspirated by pipette tip 42 here and are discharged at waste liquid disposal section (not illustrated).

Next, magnet 20 is kept away from well 7. In addition, laminate film 12 covering well 8 is perforated by a cutter part (not illustrated). And pipette part 41 forces pipette tip 42 to penetrate in well 8 and aspirates cleaning fluid 103 in well 8. Then the pipette moves, and pipette part 41 forces pipette tip 42 to penetrate in well 7 and discharges the cleaning solution aspirated from well 8 in well 7. Next, cleaning fluid and solution in well 7 are stirred with pipette tip 42, and magnet 20 is elevated to centralize magnetic particles 43 on which nucleic acid adheres in one point of the inner wall of well 7. Here, substances other than magnetic particles 43 on which nucleic acid adheres are aspirated by pipette tip 42 (making a state similar in FIG. 7) and are discharged at waste liquid disposal section (not illustrated).

Furthermore, by a similar step as described above, ethanol 104 in well 9 is moved to well 7 and stirred, and magnet 20 is elevated to centralize magnetic particles 43 on which nucleic acid adheres in one point of the inner wall of well 7. And substances other than magnetic particles 43 on which nucleic acid adheres are aspirated by pipette tip 42 (making a state similar in FIG. 7) and are discharged at waste liquid disposal section (not illustrated).

By each of the above-mentioned steps, cleaning treatment in the purification step is completed. And the process shifts to treatment to elute nucleic acid from magnetic particles 43 on which nucleic acid adheres.

Similarly as each of the above-mentioned steps, the solution in well 7 is moved to well 6 and the eluate in well 10 to well 6 using pipette device 40. Then after the mixture in well 6 is stirred and allowed to stand, and magnet 20 is elevated to centralize magnetic particles in one point of the inner wall of well 6. Since the nucleic acid is separated from the magnetic particles by eluate moved from well 10, the solution in well 6 except magnetic particles is a solution of nucleic acid obtained through purification. A predetermined amount of this nucleic acid solution is aspirated from well 6 by pipette and transferred into well 4 in which laminate film 12 is perforated by a cutter part beforehand so as to be open. Thus the purification step of this example is completed.

Next, pipette tip 42 is exchanged before shifting to the second PCR. Pipette tip 42 used in the first PCP and the purification step is disposed and new pipette tip 42 is set. That is, before the first PCR, pipette tip 42 is taken out from pipette depot 201 shown in FIG. 1B and attached to pipette part 41, and after used in the first PCR and the purification step, it is returned to the original position in pipette depot 201 or disposed to a particularly provided area for disposal. And a new pipette tip 42 is taken out to use it in the second PCR from pipette depot 201 and set it to pipette part 41.

After pipette tip 42 is exchanged, the process shifts to the second PCR (the second amplification step). First, laminate film 12 covering well 11 is perforated and new pipette tip 42 is inserted in well 11. And the solution 106 used in the second PCR filled up in well 11 beforehand is moved into well 4. And the nucleic acid solution in well 4 and the solution 106 used in the second PCR are stirred by pipette tip 42. After the stirring is finished, pipette part 41 is evacuated from reaction/storing vessel 3 and top unit 26 and lid 13 are made into a state shown in FIG. 5 as in the first PCR. And holding plate 32 is elevated with leading screw 34 so that it is in a state shown in FIG. 3. Then, the second PCR is performed which amplifies nucleic acid in a sample tube by repeating around 25 times temperature cycles of maintaining each of the temperatures of about 92° C.-55° C.-72° C. for a predetermined period of time. Since the reagents have been already consumed entirely at this time point, cooling of cooling block 24 may be stopped during the second PCR.

Another labeling process in the second PCR which generates the target nucleic acid from a labeled substrate, as mentioned in the background art also accords with the purpose of the present invention.

After the second PCR is completed in this way, top unit 26 and lid 13 are made into a state shown in FIG. 6. And the amplification product is moved to the hybridization section 207 (not illustrated) from well 4 by pipette tip 42. When this transportation is finished, the apparatus is made into a state shown in FIG. 3 again and holding plate 32 is lowered and carriage part 37 is transported forward to make reaction/storage vessel 3 removal from DNA testing apparatus.

Treatment to generate hybridization bond in hybridization section 207 is performed, and presence of hybridization bond is detected in detection section 208. Since these steps can be performed by a known conventional method and the description thereof is omitted.

As described with reference to FIG. 3, solutions used in each step are filled in reaction/storage vessel 3 beforehand in the amplifying section 206 of this example according to the order to be used. Therefore, pipette tip 42 which aspirates and holds a solution is configured so that it does not pass above the unused wells which are not opened if possible. By taking such a constitution, even if a little amount of solution drops from pipette tip 42, there is little possibility where it drops in an unused well, it does not affect the amplification step and the purification step. In addition, laminate film 12 covering each of wells 4 to 11 is perforated with a cutter part by an appropriate timing. The timing to open this hole is not limited in particular as long as it is before pipette tip 42 is forced to penetrate in each of wells 4 to 11. However, it is preferable to perforate just before the step to use the solution therein to surely prevent contamination by a drop from pipette tip 42 of each solution as described above.

In this example, pipette tip 42 is exchanged after the first PCR and purification, that is, just before the second PCP as described above and the target nucleic acid is moved to amplification solution of the second PCR using a new pipette tip 42. And the second PCP is performed and the treatment is performed using the pipette tip 42 thereafter. In addition, it can be contemplated in another example to perform the second PCR by cleaning pipette tip 42 in cleaning section 202 after the first PCR and purification, that is, just before the second PCR and moving the target nucleic acid into the amplification solution of the second PCR. There is no fear that well 4 in which the second PCR is to be performed is contaminated with a solution which has been used in the first PCR (the first and the second primers) in any of the examples described above. As a result, labeled target nucleic acid can be contained at a desired ratio, and therefore, it can be bound with probes in hybridization efficiently. In addition, since nucleic acids complementary to the target nucleic acid is not amplified in the second PCR, labeled target nucleic acid is not consumed in vain by hybridization in liquid. Therefore, it is possible to allow the labeled target nucleic acid to be bound to probes in hybridization efficiently.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2005-291181, filed Oct. 4, 2005 which is hereby incorporated by reference herein in its entirety.

1-11. (canceled)

12. A biochemical treatment process comprising the following steps:
a first amplification step in which a target nucleic acid and a nucleic acid complementary to the target nucleic acid are amplified;
a purification step in which substances other than the nucleic acids amplified in the first amplification step are removed;
a second amplification step in which a labeled target nucleic acid is amplified using the purified nucleic acids; and
a step of exchanging or cleaning at least a part of a liquid handling mechanism for transporting a liquid containing the nucleic acids amplified in the first amplification step which part is in contact with the liquid between the first amplification step and the second amplification step.

13. The biochemical treatment process according to claim 12, wherein the liquid is handled by using a pipette device as the liquid handling mechanism and a pipette tip in contact with the liquid is exchanged or cleaned in the exchanging or cleaning step.

14. The biochemical treatment process according to claim 12, wherein the part in contact with the liquid is continued to be used without exchange or cleaning in the first amplification step and the purification step.

15. The biochemical treatment process according to claim 12, wherein the amplification in the first and second amplification steps is conducted by PCR treatment.

16. The biochemical treatment process according to claim 13, wherein the first amplification step and the purification step are performed by a first pipette tip, and wherein the first pipette tip is exchanged into a second pipette tip at the step of exchanging or cleaning.

17. The biochemical treatment process according to claim 12, further comprising a step of extracting a nucleic acid from a biological sample before the first amplification step, wherein the nucleic acid extracted by the step of extracting is subjected to the first amplification step.

18. A biochemical treatment process using a pipette device to which a pipette tip can be attached comprising the following steps:
a first amplification step in which a target nucleic acid and a nucleic acid complementary to the target nucleic acid are amplified using a first pipette tip;
a purification step in which substances other than the nucleic acids amplified in the first amplification step are removed using the first pipette tip;
a step of exchanging the first pipette tip into a second pipette tip after the purification step; and
a second amplification step in which a labeled target nucleic acid is amplified using the purified nucleic acids by using the second pipette tip.

19. A biochemical treatment process using a pipette device comprising the following steps:
a first amplification step in which a target nucleic acid and a nucleic acid complementary to the target nucleic acid are amplified using the pipette device;
a purification step in which substances other than the nucleic acids amplified in the first amplification step are removed using the pipette device which is not cleaned between the first amplification step and the purification step;
a step of cleaning the pipette device after the purification step;
a second amplification step in which a labeled target nucleic acid is amplified using the cleaned pipette device.
